(19) United States
(12) Patent Application Publication
(10) Pub. No.: US 2026/0041720 A1
(43) Pub. Date: Feb. 12, 2026

(54) BIFIDOBACTERIUM LONGUM AND METHOD OF DECREASING BLOOD TMAO AND INCREASING THE CONTENT OF AKKERMANSIA MUCINIPHILA IN INTESTINES THEREOF

(71) Applicant: Leeuwenhoek Laboratories Co. Ltd., Taipei City (TW)

(72) Inventors: Cheng-Chih HSU, Taipei City (TW); Chi-Shan LI, Taipei City (TW); Chiao-Wei LIN, Taipei City (TW); Yi-Ling LO, Taipei City (TW); Hsin-Bai ZOU, Taipei City (TW)

(21) Appl. No.: 19/090,425

(22) Filed: Mar. 26, 2025

(30) Foreign Application Priority Data
Aug. 6, 2024 (TW) 113129462

(51) Int. Cl. A61K 35/745; A61K 9/00; A61P 1/00; A61P 9/00; C12N 1/20

(52) U.S. Cl. CPC A61K 35/745; A61K 9/0053; A61P 1/00; A61P 9/00; C12N 1/205

(57) ABSTRACT

A Bifidobacterium longum strain LWHK1005 is provided, and the strain prevents and treats cardiovascular diseases and gastrointestinal diseases at the same time, and has the function of reducing trimethylamine-N-oxide in the blood and increasing the content of Akkermansia muciniphila in the intestines. A method of decreasing blood TMAO and increasing a content of Akkermansia muciniphila in intestines comprising administering to a subject in need thereof an effective amount of bacteria strain as above mentioned is also provided.

### CROSS-REFERENCE TO RELATED APPLICATION

This application claims priority to Taiwan Application Serial Number 113129462, filed on Aug. 6, 2024, which is herein incorporated by reference in its entirety.

The Sequence Listing associated with this application is filed in electronic format via EFS-Web and is hereby incorporated by reference into the specification in its entirety. The name of the XML file containing the Sequence Listing is NP-36403-US_SEQ_LIST.xml. The size of the XML file is 5,218 bytes, and the XML file was created on Feb. 24, 2025.

### BACKGROUND

### Field of Invention

The present invention relates to a Bifidobacterium longum and method thereof. More particularly, the present invention relates to a Bifidobacterium longum and use of decreasing blood trimethylamine-N-oxide (TMAO) and increasing the content of Akkermansia muciniphila in intestines thereof.

### Description of Related Art

Cardiovascular diseases and gastrointestinal diseases are the most common diseases currently. Cardiovascular disease refers to diseases of the heart and blood vessels. The cause of cardiovascular disease is usually atherosclerosis, which leads to blood vessel blockage and blood flow disorders, resulting in symptoms such as palpitations, difficulty breathing, and dizziness. Gastrointestinal diseases generally refer to symptoms or diseases of inflammation and ulcers occurring in the digestive organs, and new treatments are urgently needed.

“Probiotics” are active microorganisms that coexist with a host. Appropriate intake of probiotics can have a beneficial effect on the host's health. Although traditional probiotics such as Lactobacillus and Bifidobacterium have been widely used and have good safety and human tolerance, the effects on human intestinal microorganisms are still limited, and there are great differences in the benefits and mechanisms between different strains.

Therefore, how to provide probiotics that can treat both cardiovascular diseases and gastrointestinal diseases at the same time, the related art really needs to be improved.

### SUMMARY

The present disclosure provides a Bifidobacterium longum LWHK1005 deposited at DSMZ under number DSM 35052.

The present disclosure also provides a composition for decreasing blood TMAO and increasing a content of Akkermansia muciniphila in intestines, comprising: the Bifidobacterium longum LWHK1005 as above mentioned; and a pharmaceutically acceptable carrier.

In some embodiments, the Bifidobacterium longum LWHK1005 is a live strain.

In some embodiments, the pharmaceutically acceptable carrier is selected from the group consisting of micro-crystalline cellulose, trehalose, maltodextrin, rice flour, magnesium stearate, inositol, dextrose, sucrose, and a combination thereof.

The present disclosure also provides a method of decreasing blood TMAO and increasing a content of Akkermansia muciniphila in intestines comprising administering to a subject in need thereof an effective amount of bacteria strain, wherein the bacteria strain is Bifidobacterium longum LWHK1005 as above mentioned.

In some embodiments, the bacteria strain is live strain.

In some embodiments, the decreasing blood TMAO is to prevent cardiovascular disease, prevent chronic kidney disease, or a combination thereof.

In some embodiments, the bacteria strain increase the content of Akkermansia muciniphila in intestines to increase intestinal barrier function, increase immunity, reduce weight, reduce fat weight, inhibit inflammation, lower blood sugar, lower blood lipids, inhibit fatty liver and fatty liver hepatitis, improve insulin resistance, or a combination thereof.

In some embodiments, the bacteria strain is in a form of capsule, tablet, powder or liquid.

In some embodiments, the bacteria strain is formulated for oral delivery.

In some embodiments, the bacteria strain is administered as a drug or a health supplement.

In some embodiments, the drug comprises a pharmaceutically acceptable carrier.

In some embodiments, the pharmaceutically acceptable carrier is selected from the group consisting of micro-crystalline cellulose, trehalose, maltodextrin, rice flour, magnesium stearate, inositol, dextrose, sucrose, and a combination thereof.

In some embodiments, the health supplement comprises a food acceptable carrier.

In some embodiments, a bacteria count of the bacteria strain is from 5×10⁸ CFU to 1×10⁹ CFU, for example, 5×10⁸ CFU, 6×10⁸ CFU, 7×10⁸ CFU, 8×10⁸ CFU, 9×10⁸ CFU, 1×10⁹ CFU, or any value between any two of these values.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIG. 1 is the difference in reducing TMAO by different strains according to one embodiment of the present disclosure.

FIG. 2 is the changes in the content of Akkermansia muciniphila in the intestine caused by different strains according to one embodiment of the present disclosure.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

Bifidobacterium longum (B. longum) is a Gram-positive, catalase-negative, rod-shaped bacterium found in the human gastrointestinal tract. It is an anaerobic bacterium and is considered to be one of the earliest bacteria present in the infant gastrointestinal tract.

The strain of Bifidobacterium longum LWHK1005 of present disclosure has been deposited at Leibniz Institute DSMZ-German Collection of Microorganisms and Cell Cultures GmbH (address of depositary institution: Inhoffenstr. 7B, D-31824 Braunschweig, Germany) on Jun. 14, 2024, and the deposit number is DSM 35052. The strain of Bifidobacterium longum LWHK1005of present disclosure also has been deposited at bioresource collection and research center (BCRC) in Taiwan (BCRC) (address of depositary institution: No. 331 Shih-Pin Road, Hsinchu, Taiwan 300.) on Apr. 16, 2024, and the deposit number is BCRC 911220.

The Bifidobacterium longum LWHK1005 disclosed in the present disclosure is an anaerobic bacterium isolated from the feces of healthy humans in Taiwan. The strain is rod-shaped with Y-shaped branches and is approximately 4 micrometers (μm) to 8 μm in length. The live Bifidobacterium longum disclosed in the present disclosure was cultured in MRS culture medium (dE Man, Rogosa and Sharpe broth) under anaerobic conditions at 37° C. The 16S rRNA gene sequence is shown in SEQ ID NO: 1.

In some embodiments of the present disclosure, the strain is administered to a subject orally or parenterally. In some embodiments of the present disclosure, the strain is formulated into an oral dosage form selected from the group consisting of solution, suspension, emulsion, powder, lozenge, pill, syrup, buccal lozenge, tablet, chewing gum and capsule for administration to a subject.

In some embodiments, the pharmaceutically acceptable carrier comprises, but is not limited to water, alcohols, glycol, preserving agents, antioxidants, solvent, emulsifier, suspending agent, decomposer, binding agent, excipient, stabilizing agent, chelating agent, diluent, gelling agent, preservative, lubricant, absorption enhancers, active agents, humectants, odor absorbers, fragrances, pH adjusting agents, occlusive agents, emollients, thickeners, solubilizing agents, penetration enhancers, anti-irritants, colorants, propellants, surfactant, and other similar or applicable carriers for the present invention.

As used herein, the term “cardiovascular disease” refers to diseases of the heart and blood vessels. The common cause is atherosclerosis leading blood vessel blockage and blood flow problems, thereby causing a variety of symptoms such as palpitations, dyspnea, cyanosis, dizziness, syncope, etc. Common cardiovascular diseases include coronary heart disease, stroke, heart failure, arrhythmia, valvular heart disease, but the present disclosure is not limited thereto. Once diagnosed, treatment options include medication and coronary artery surgery including balloon angioplasty, heart stents, and coronary artery bypass surgery.

As used herein, the term “gastrointestinal diseases” includes symptoms or diseases such as inflammation and ulcers occurring in the digestive organs. For example, gastrointestinal diseases include aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease, but the present disclosure is not limited thereto.

As used herein, the term “subject” refers to a warm-blooded animal, such as a mammal suffering from a particular disease, disorder or illness, and includes, for example, humans, orangutans, chimpanzees, mice, rats, dogs, cows, chickens, pigs, goats, sheep, etc., but the disclosure is not limited thereto.

As used herein, the term “treatment” or “treating” includes alleviating symptoms, temporarily or permanently removing the cause of symptoms, avoiding or slowing the occurrence of symptoms and the progression of a disease, disorder or discomfort, but the disclosure is not limited thereto.

As used herein, the term “Akkermansia muciniphila (A. muciniphila)” is an extremely anaerobic Gram-negative bacterium. It is one of the most abundant species in the human intestinal microorganisms, accounting for about 1-5% of the total number of bacteria in the human intestinal tract. It decomposes mucin as the only source of carbon, nitrogen and energy, and produces short-chain fatty acids (SCFAs) such as acetate and propionate. Improving obesity is the first confirmed health benefit of A. muciniphila (Everard et al., 2013). Obesity is associated with chronic low-grade inflammation, which is related to specific changes in the gut microbiota (Plovier et al., 2017). In obese individuals, there is an increase in the abundance of Firmicutes in the gut, while the abundance of A. muciniphila is significantly reduced. Additionally, the abundance of A. muciniphila is negatively correlated with body fat weight and impaired glucose tolerance. It has been found that A. muciniphila can also improve indicators related to metabolic syndrome. A. muciniphila can mitigate metabolic abnormalities induced by a high-fat diet, including increased fat mass, adipose tissue inflammation, and insulin resistance.

As used herein, the term “TMAO” is also called trimethylamine-N-oxide (TMAO), which is the N-oxide formed by trimethylamine. The chemical formula is (CH₃)₃NO. It is a metabolite formed by intestinal anaerobic bacteria and the human body. In recent years, it has been considered an important factor leading to cardiovascular disease. TMAO promotes the accumulation of macrophages in the blood vessel walls, inhibits the cholesterol recycling pathway, and enhances platelet aggregation activity, which may lead to the formation of atherosclerosis and embolism and the increase of the risk of heart disease.

A number of examples are provided herein to elaborate the Bifidobacterium longum and use of decreasing blood TMAO and increasing the content of Akkermansia muciniphila in intestines thereof of the present disclosure. However, the examples are for demonstration purpose alone, and the instant disclosure is not limited thereto.

### Example

### Experimental Group

In this experiment, 8-week-old C57BL/6 female mice were used and randomly divided into 1 control group (Con) and 6 experimental groups. The experimental groups were gavaged with different live strains of Bifidobacterium longum (A1, A2, A3, A4, LWHK1005, A6) daily. The bacterial count was 2×10⁸ CFU and was suspended in phosphate buffered solution (PBS) containing 2.5% glycerol. The control group was gavaged with an equal volume of PBS containing 2.5% glycerol daily. The gavaged period is six weeks.

### Example 1

In order to understand the differences in the efficacy of different Bifidobacterium longum strains in inhibiting the production of TMAO in mice, the mice were given choline chloride (some bacteria can metabolize choline into TMA, which will enter the liver through the blood and be metabolized into TMAO), and the concentration of TMAO in the mouse blood was detected. The experimental procedure was as follows: different Bifidobacterium longum strains (A1, A2, A3, A4, LWHK1005, A6) were gavaged daily for six weeks. Next, the mice were fasted for 12 hours, and then blood was collected after fasting (this was the 0-hour blood sample). Then, the mice were given choline chloride (dissolved in water) at a dose of 400 mg/kg BW. Four hours after the mice were gavaged with choline chloride, blood was collected (this was the 4-hour blood sample). After plasma was separated from the blood samples, the TMAO content in the samples was analyzed using liquid chromatography mass spectrometry (LC-MS).

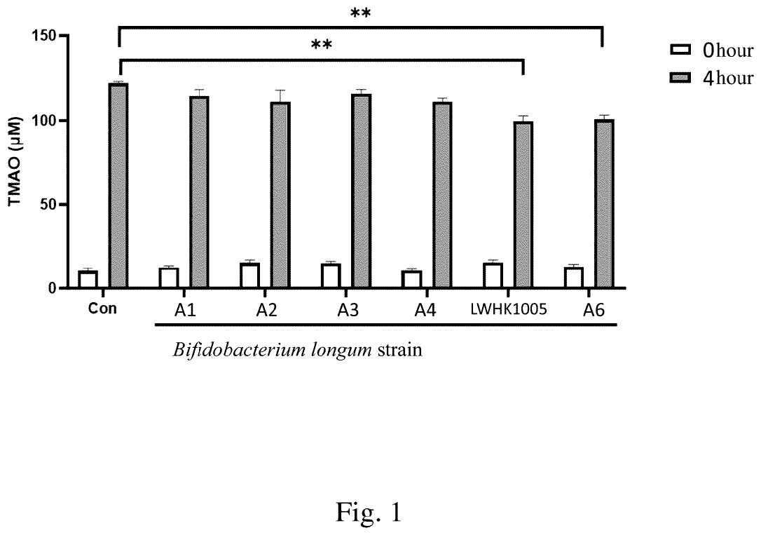

The results are shown in FIG. 1, which compares the blood TMAO concentrations of each group before (0 hour) and after (4 hours) choline chloride ingestion. Before administration of choline chloride, the blood TMAO concentration was 10.427±1.603 μM (Mean±SEM) in the control group, 12.730±0.974 μM in the Bifidobacterium longum strain A1 group, 15.354±1.909 μM in the Bifidobacterium longum strain A2 group, 15.193±1.249 μM in the Bifidobacterium longum strain A3 group, 10.638±0.884 μM in the Bifidobacterium longum strain A4 group, 15.646±1.552 μM in the Bifidobacterium longum LWHK1005 group, and 12.808±1.813 μM in the Bifidobacterium longum strain A6 group. After statistics, there was no statistical difference between the groups. Four hours after administration of choline chloride, the blood TMAO concentration was 121.968±1.089 μM in the control group, 114.568±3.662 μM in the Bifidobacterium longum strain A1 group, 111.051±6.838 μM in the Bifidobacterium longum strain A2 group, 115.674±2.677 μM in the Bifidobacterium longum strain A3 group, 111.164±2.056 μM in the Bifidobacterium longum strain A4 group, 99.581±3.267 μM in the Bifidobacterium longum LWHK1005 group, and 100.687±2.575 μM in the Bifidobacterium longum strain A6 group. After mice were given choline chloride, the blood TMAO concentration of all groups of mice increased significantly. However, in the groups gavaged with Bifidobacterium longum LWHK1005 or strain A6, the blood TMAO concentrations after administration of choline chloride were significantly lower than that of the control group (t-test, **: p<0.01).

### Example 2

To compare the differences in the intestinal content of a second-generation probiotic Akkermansia muciniphila between different Bifidobacterium longum strains, fecal samples of mice were collected after being gavaged with different Bifidobacterium longum strains (A1, A2, A3, A4, LWHK1005, A6) for six weeks. DNA was extracted from fecal samples, and the Akkermansia muciniphila contents in fecal samples were analyzed by real-time polymerase chain reaction (RT-PCR) using a primer pair designed for Akkermansia muciniphila ATCC BAA-835 strain (SEQ ID NO: 2-forward primer CAGCACGTGAAGGTGGGGAC, SEQ ID NO: 3-reverse primer CCTTGCGGTTGGCT TCAGAT).

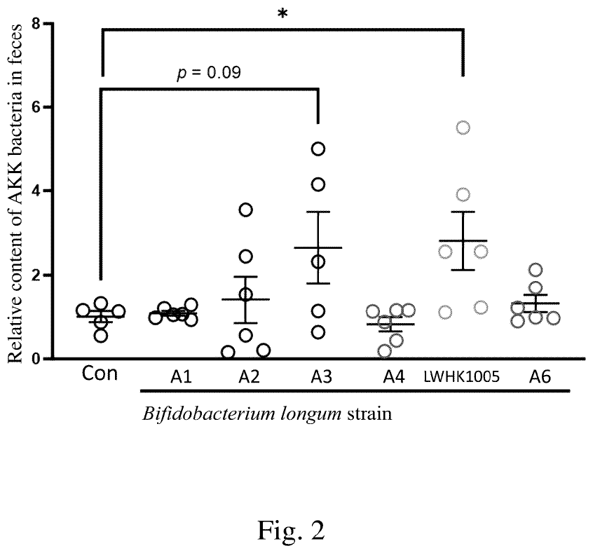

After six weeks of daily gavaged with different Bifidobacterium longum strains (A1, A2, A3, A4, LWHK1005, A6), the levels of Akkermansia muciniphila in the fecal samples of the mice were analyzed to understand whether the Akkermansia muciniphila in their intestines was changed by the administration of different Bifidobacterium longum strains. The analysis results are shown in FIG. 2. Compared with the control group, the Akkermansia muciniphila content in the Bifidobacterium longum strain A1 group was 1.078±0.055 folds (Mean±SEM), the Bifidobacterium longum strain A2 group was 1.406±0.56 folds, the Bifidobacterium longum strain A3 group was 2.651±0.846 folds, the Bifidobacterium longum strain A4 group was 0.818±0.17 folds, the Bifidobacterium longum strain LWHK1005 group was 2.812±0.686 folds, and the Bifidobacterium longum strain A6 group was 1.312±0.203 folds. The content of Akkermansia muciniphila in fecal samples of the group gavaged with Bifidobacterium longum strain LWHK1005 was significantly higher than that of the control group (t-test, *: p<0.05). Although there was no statistical difference in the groups gavaged with Bifidobacterium longum strains A2 and A3, the contents of Akkermansia muciniphila have higher trend than that in the control group.

It is worth noting that not all Bifidobacterium longum strains can increase the content of Akkermansia muciniphila. For example, the Akkermansia muciniphila contents in the Bifidobacterium longum strains A1 and A4 and the control group were similar.

The Bifidobacterium longum strain LWHK1005 disclosed in the present invention has the functions of inhibiting MTAO and increasing the content of Akkermansia muciniphila, and has the functions of reducing TMAO in the blood and increasing the content of Akkermansia muciniphila in the intestine at the same time, thereby preventing or treating cardiovascular diseases and gastrointestinal diseases.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A Bifidobacterium longum LWHK1005 deposited at DSMZ under number DSM 35052.

2. A composition for decreasing blood TMAO and increasing a content of Akkermansia muciniphila in intestines, comprising:
the Bifidobacterium longum LWHK1005 as claimed in claim 1; and
a pharmaceutically acceptable carrier.

3. The composition of claim 2, wherein the Bifidobacterium longum LWHK1005 is a live strain.

4. The composition of claim 2, wherein the pharmaceutically acceptable carrier is selected from the group consisting of micro-crystalline cellulose, trehalose, maltodextrin, rice flour, magnesium stearate, inositol, dextrose, sucrose, and a combination thereof.

5. A method of decreasing blood TMAO and increasing a content of Akkermansia muciniphila in intestines comprising administering to a subject in need thereof an effective amount of bacteria strain, wherein the bacteria strain is Bifidobacterium longum LWHK1005 as claimed in claim 1.

6. The method of claim 5, wherein the bacteria strain is live strain.

7. The method of claim 5, wherein the decreasing blood TMAO is to prevent cardiovascular disease, prevent chronic kidney disease, or a combination thereof.

8. The method of claim 5, wherein the bacteria strain increase the content of Akkermansia muciniphila in intestines to increase intestinal barrier function, increase immunity, reduce weight, reduce fat weight, inhibit inflammation, lower blood sugar, lower blood lipids, inhibit fatty liver and fatty liver hepatitis, improve insulin resistance, or a combination thereof.

9. The method of claim 5, wherein the bacteria strain is in a form of capsule, tablet, powder or liquid.

10. The method of claim 5, wherein the bacteria strain is formulated for oral delivery.

11. The method of claim 5, wherein the bacteria strain is administered as a drug or a health supplement.

12. The method of claim 11, wherein the drug comprises a pharmaceutically acceptable carrier.

13. The method of claim 12, wherein the pharmaceutically acceptable carrier is selected from the group consisting of micro-crystalline cellulose, trehalose, maltodextrin, rice flour, magnesium stearate, inositol, dextrose, sucrose, and a combination thereof.

14. The method of claim 11, wherein the health supplement comprises a food acceptable carrier.

15. The method of claim 5, wherein a bacteria count of the bacteria strain is from 5×10⁸ CFU to 1×10⁹ CFU.


SEQUENCE LISTING (3 sequences)

SEQ ID NO: 1 - DNA, 1443 bp, Bifidobacterium longum
  source 1..1443: mol_type = other DNA
agtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcagtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaa

SEQ ID NO: 2 - DNA, 20 bp, synthetic construct
  source 1..20: mol_type = other DNA
cagcacgtgaaggtggggac

SEQ ID NO: 3 - DNA, 20 bp, synthetic construct
  source 1..20: mol_type = other DNA
ccttgcggttggcttcagat